(19) United States
(12) Patent Application Publication
(10) Pub. No.: US 2009/0214434 A1
(43) Pub. Date: Aug. 27, 2009

(54) METHOD OF DETECTING AND TREATING ALLOGENIC CELLS RESPONSIBLE FOR ENDOMETRIOSIS

(76) Inventor: Dennis Hardy O'Dowd, Toronto (CA)

Correspondence Address: KLARQUIST SPARKMAN, LLP, 121 SW SALMON STREET, SUITE 1600, PORTLAND, OR, 97204, US

(21) Appl. No.: 11/574,003

(22) PCT Filed: Aug. 19, 2005
(86) PCT No.: PCT/CA2005/001269

§ 371 Date: Feb. 16, 2009

Related U.S. Application Data
(60) Provisional application No. 60/602,888, filed on Aug. 20, 2004.
(60) Provisional application No. 60/679,672, filed on May 11, 2005.

(51) Int. Cl. A61K 49/00

(52) U.S. Cl. 424/9.6

(57) ABSTRACT

There are presently provided methods for identifying an ectopic allogenic cell in a mammalian host, the methods comprising obtaining a first internal soft tissue image, administering an effective amount of iodine to potentiate the host's immune response to aberrant ectopic tissue, obtaining one or more subsequent internal soft tissue images after said administration and comparing the first internal soft tissue image and the one or more subsequent internal images to identify the ectopic allogenic cell. The methods can also be used for determining or improving the efficiency of treatment of pathology disease or disorder caused by the presence of allogenic commensal or commensal-like cells. Furthermore, the methods can be used to diagnose, and also treat, endometriosis.

### CROSS-REFERENCE TO RELATED APPLICATIONS

The present application claims the benefit of prior provisional applications Ser. No. 60/602,888, filed Aug. 20, 2004, and Ser. No. 60/679,672, filed May 11, 2005, the contents of which provisional applications are hereby incorporated herein by reference.

### FIELD OF THE INVENTION

The present invention relates generally to the identification of allogenic cells in the body responsible for a pathology including endometriosis and the treatment of the same.

### BACKGROUND OF THE INVENTION

Endometriosis is characterized by the ectopic presence of cells containing endometrial glands and stroma. Endometriosis is estimated to affect up to 13.6 million women in North America (and up to 10% of women worldwide) and can cause significant pain and suffering. The severity of the symptoms of endometriosis may not correlate to the size of endometrial lesions: patients with the least aberrant tissue involvement may have the most severe symptoms whereas patients with extensive ectopic endometrial tissue may be asymptomatic (Hill J A, et al. in Fertil. Steril. (1988) 50: 216-222).

The incidence of endometriosis is increasing and is approaching background reproduction rates of some populations. Endometriosis is a strongly negative factor in successful reproduction and is implicated in affecting 25 to 50% of infertile women, suggesting that the root cause of endometriosis is unlikely to be genetic, notwithstanding a statistical analysis of an Icelandic population suggesting it is (Stefanson et al., Pub. Hum Reprod. 2002, 17(3):559), unless it confers a stronger benefit than its liability. A number of theories have attempted to explain the etiology of endometriosis, including the metaplasia theory (Koninckx et al (1999) Gynecol. Obstet. Invest. 47:3), the congenital theory, and the vascular theory (http://www.centerforendo.com/QandA.htm). However, the most widely accepted theory postulates endometriosis results from retrograde menstruation enabling detached endometrial cells to implant under membrane surfaces within the peritoneal cavity (Leyendecker et al. Pub. Hum Reprod (2002), 17(3):555) and elsewhere in the body, including under the membrane tissues of the vagina. This theory, however, cannot account for endometriosis found in women without patent fallopian tubes (hence no pathway from the uterus to the peritoneal cavity), or in individuals without active endometrial tissues (Matarese et al, (2003) Trends in Mol. Med. 9(5):223; Marsh and Laufer, (2005) Fertil. Steril. 83(3):758-60) such as in infants, pre-menstrual girls, adult males treated with oestrogen, or women after having undergone a complete hysterectomy. Endometriosis re-occurs even after a full hysterectomy and removal of the ovaries approximately 40% of the time in the first five years post surgery, (“What is Radical Surgery for Endometriosis”, Well Connected Reports, University of Maryland Medicine, www.umm.edu) which implies a causative endometrial stratum basale mother cell population independent of the uterus elsewhere in the body. Rupture of an endometrioma is unpredictable and this condition can be a threat to both life and continued fertility.

Over her lifetime, a woman may move from Stage 1 endometriosis (a large asymptomatic volume of exogenous endometrial implants and a volume of immune cells and activity 3 to 6 times normal) to Stage IV, (a small volume of exogenous endometrial implants initiating autoimmune reactions including spontaneous autoimmune abortion, (“Pathenogenesis of Endometriosis, natural immunity dysfunction or autoimmune disease” Giuseppe Matareses, et al, Trends in Molecular Medicine, VOL 9, No. 5, May 2003) from a normal to less than normal volume of immune cells (Hill J A, et al “Characterization of leucocyte subpopulations in the peritoneal fluid of women with endometriosis”, in Fertil Steril, 1988; 50 216-222), as a result of her immune system resolving these endometriomas without treatment.

The self-resolution of existing endometrial implants forming endometriomas between Stage I and Stage IV, is resolution only of non-commensal daughter endometrial stratum functional cells (ESF hereinafter), and not of self-motile, commensal-like endometrial stratum basale mother cells (ESB hereinafter) of this condition (Ferencxy, et al. “Studies on the Cytodynamics of Human Endometrial Regeneration”, AM. Jour. Of Obstet, Gyn, 1979, June, 1:134(3):297-304). Currently, it is believed that ESB cells only reproduce themselves when extending a single cell layer cover of themselves over muscle tissue of the uterus during uterine repair, or may do so when implanted on organ muscle tissue exogenously.

This self-resolution involves successful antigen presentation despite iron homeostasis dis-equilibration (“The influence of iron homeostasis on macrophage function” Ward, J. et al, Biochemical Society Transactions, [2002] Vol 30, part 4, p 726). As a result of this resolution, those particular commensal-like ESB cells will not be able to successfully produce non commensal-like ESF daughter cells, without those ESF cells attracting attention from the cell-mediated immune (“CMI”) system. Thus, such cells remain covert, staying in place or relocating in the body, except where natural, patient-induced (i.e. pregnancy, or by consumption of anti-oxidants) or iatrogenic immuno-down regulation of the CMI is established by various means, for instance and including surgical removal of large volumes of tissue resulting in the simultaneous removal of local resident macrophages, or consumption of excessive amounts of various antioxidants including vitamin E and/or dietary zinc or by other such products and iatrogenic means known to those with ordinary skill in the art. Both uterine fibroids and endometriomas most often tend to resolve by first breaking up into smaller units, which effect is suggestive of there being more than one condition-causative, self-motile and reproductively competent initiator cell present in those larger endometriomas and fibroids. Both such tissue masses also behave similarly under iatrogenically expedited growth, re-consolidating into larger units.

The current state of the art in identifying the numbers and locations of these ESB cells is without any real assurance of their actual presence, location and numbers, resulting in removal of healthy tissue in the hope of reducing the number of further corrective surgeries needed to remove further ESB cells.

A number of treatments have been proposed for endometriosis, however, no cure for endometriosis has yet emerged. Surgical removal of identified endometriomas is only a temporary measure: after treatment endometriosis reoccurs in most women. Merck Manual, Second Home Edition, Chapter 245. Given the current uncertainty as to the source of ectopic endometrial tissue, surgery suffers from the drawback that while it may remove identifiable endometriomas, it may not remove all cells that are responsible for endometriosis. Laparoscopy, which results in a correct diagnosis approximately 90% of the time (Dmowski et al, Fertil and Steril 67:238), may also be used to remove ectopic endometrial tissue. In more severe cases, for instance where endometrial tissues are 1.5 to 2 inches (3.8 to 5.1 cm) in diameter or where endometrial tissue blocks one or both fallopian tubes, more extensive abdominal surgery may be required. (Merck Manual, Second Home Edition, Chapter 245.)

### SUMMARY OF THE INVENTION

The invention is predicated in part on the discovery that the presence of allogenic cells in the body is responsible for pathologies, including endometriosis, and that an excess of degraded heme produced by the daughter cells of these allogenic cells may block effective phagocytosis and antigen presentation by macrophages. The invention, in different aspects, provides methods of identifying these allogenic cells and of treating a pathology, disease, or disorder related to, or caused by the presence of such cells, including endometriosis, and a means of evaluating the efficacy of such treatment(s). While endometriosis presents a model disease, it will be appreciated by those persons skilled in the art reading this specification that the principles and methods of the invention can be applied to other diseases or pathologies related to or caused by the presence of allogenic commensal-like cells and the identification of allogenic commensal-like cells, as for example, endometrial stratum basale cells.

In one aspect, there is provided a method for identifying an ectopic allogenic initiator cell in a mammalian host, the method comprising obtaining a first internal soft tissue image; administering an effective amount of iodine to potentiate the host's immune response to aberrant ectopic tissue; obtaining one or more subsequent internal soft tissue images after the administration; and comparing the first internal soft tissue image and the one or more subsequent internal images to identify the position of the ectopic allogenic cell.

The allogenic initiator cell is, in one embodiment, an endometrial stratum basale cell (ESB).

In another aspect, there is provided a method for identifying an allogenic ectopic causative cell, the method comprising obtaining a first internal soft tissue image; administering an effective amount of iodine to potentiate the host's immune response to aberrant ectopic tissue; obtaining one or more subsequent internal soft tissue images after said administration; and comparing the first internal soft tissue image and the one or more subsequent internal images to identify the position of the allogenic ectopic causative cell.

The allogenic causative cell is, in one embodiment, an endometrial stratum functional cell (ESF).

The iodine is administered to potentiate the host's immune response to, and clean up of, aberrant ectopic tissue, tissue remains, and degraded heme, thereby reducing the volume of an endometrioma so that monthly re-growth is detectable. This method can also be used to identify an iatrogenic ectopic allogenic ESB cell, for example, under conditions where antioxidants permit the establishment of endometriomas, or re-establishment of unresolved endometriomas. This method may be performed without additional hazard to the patient as any endometriomas enlarged can be shrunk quickly.

In a further aspect there is provided a method of identifying a covert ectopic allogenic initiator cell whose daughter allogenic causative cells have been resolved by a patient's immune system or other treatment, by use of down-regulators of cell-mediated immunity to permit for instance, those allogenic initiator cells to express daughter causing cells, and so identifying the current location of these covert ectopic allogenic initiator cells for surgical excision, after observing this growth/recession and re-growth cycle. This method of inducing allogenic initiator cells to express allogenic causative cells without the need to wait years is useful at any time post surgery to demonstrate removal of such cells, as well as during diagnosis to evaluate the real extent and number of such cells in this and other similar conditions.

In one embodiment, the iodine is administered with an adjuvant such as an iron chelating agent, for example, desferrioxamine or o-phenanthroline (Harhaji et al., (2004) Clin Exp Immunol 137(1):109-116) or with apoptosis mediators, anti-platelet and anti-inflammatory agents such as acetylsalicylic acid (aspirin). Chelating agents may reduce the concentration of extracellular iron or change the chemical reactivity potential of extracellular iron. The iron chelators may reduce the inhibitory effects of extracellular iron on immune cell production of strong oxidizers used as or to create biocidal compounds and to “digest” engulfed cells and cell debris to provide antigens presentable to the humoral immune system and trainable cytotoxic cells. Apoptosis mediators such as, for example aspirin, may extend the time between menstrual periods (without the concurrent risk and physiological and/or psychological effects of chemical de-feminization) in which macrophage iron scavenging can take place, improving successful antigen presentation and dealing with pain and inflammation, as well as reducing blood clotting as an anti-platelet agent.

In one embodiment, the effective amount of iodine is from about 0.1 to about 28 mg/day per 50 kilograms of body weight in excess of the host's normal body and dietary requirements.

In different embodiments of the above aspects of the invention, the ectopic allogenic initiator cell is an endometrial stratum basale cell (ESB cell) and the ectopic allogenic causative cell is an endometrial stratum functional cell (ESF cell), which may form or be contained in an endometrioma.

In another aspect there is provided a method for treating endometriosis comprising identifying the location of an ectopic allogenic endometrial stratum basale cell according to the method described above and removing the ectopic allogenic endometrial stratum basale cell.

In different embodiments, removing the allogenic endometrial stratum basale cell comprises adding a bio-irritant thereby irritating or damaging the cell in a manner sufficient to induce an effective immune response against the cell or removing one or more ectopic allogenic endometrial stratum basale cells from the host, stimulating a cell-mediated immune response ex vivo by reacting the allogenic cell with the host's immune cells and reintroducing the host's immune cell into the host's body to effect a cell-mediated immune response against the allogenic stratum basale cells in the host.

In yet another aspect, there is provided a method of diagnosing endometriosis in a mammalian host, the method comprising comparing the host's genotype to the genotype of an ectopic allogenic initiator cell, which in a certain embodiment is an ESB cell.

In yet a further aspect, there is provided A method for determining or improving the efficacy of treatment of a pathology, disease or disorder caused by the presence of allogenic commensal or commensal-like cells, comprising subsequent to treatment, obtaining a first internal soft tissue image; administering an effective amount of iodine to potentiate the host's immune response to aberrant ectopic tissue; obtaining one or more subsequent internal soft tissue images after said administration; and comparing the first internal soft tissue image and the one or more subsequent internal images to determine the position of a remaining ectopic allogenic initiator cell or ectopic allogenic causative cell.

In still a further aspect, there is provided a method of determining or improving the efficacy of treatment of a pathology, disease or disorder related to or caused by the presence of allogenic commensal or commensal-like cells, comprising administering an effective amount of a down-regulator of cell-mediated immunity. Down regulation of the cell-mediated immune system using the immuno-down regulator is believed to have the effect of allowing division and reproduction of daughter cells, for example, ESF cells, of a covert ESB cell such that administration prior to treatment, for example before surgery, can improve treatment by revealing such ESB and ESF cells. As described herein, such covert cells can be identified by administering an effective amount of an immuno-down regulator, for example an anti-oxidant, followed, after an endometrioma is formed, by administering an effective amount of iodine and comparing a first internal soft tissue image obtained before iodine administration and one or more subsequent internal soft tissue images obtained after iodine administration, to identify the location of any ectopic allogenic ESB cells before treatment such as surgery. Similarly, efficacy of treatment, for example surgical removal, can be determined by this method by administering an immuno-down regulator such as an antioxidant and observing growth of new foci from any covert ESB mother cells remaining after surgery which are capable of producing daughter cells. It is believed that restarting the iodine treatment after immuno-down regulation is ceased allows for macrophage clean up of apoptotic cell debris and promotes resolution of any newly revealed covert endometriomas and ESF daughter cells, making this procedure relatively safe for the patient while providing the surgeon with a more precise target location for any such ESB cells, as described herein.

There is also presently provided various uses of an effective amount of iodine that is effective to potentiate the host's immune response to aberrant ectopic tissue, including use for identifying an ectopic allogenic initiator cell, use for identifying an ectopic allogenic causative cell, use for treating endometriosis, use for determining or improving the efficacy of treatment of a pathology, disease or disorder caused by the presence of allogenic commensal or commensal-like cells, and use for diagnosing endometriosis in a mammalian host.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of example only, embodiments of the present invention,

FIG. 1 illustrates the relationship of individual hosts to the initial chimeric forming incident and depicts the pattern of inter-generational transmission in endometriosis; and

FIG. 2 illustrates the patterns of resolution of endometrioma and uterine fibroids using thermodynamically free iodine (TFI) and TFI plus a mediator of apoptosis, such as aspirin, (TFI²) chemotherapy, and the endometrioma permissive effects associated with the minimal use of CMI immuno-down regulators as counter chemotherapy in the adult human female under close medical supervision.

### DETAILED DESCRIPTION

“Allogenic” refers to cells or tissues from the same species that are genetically dissimilar from the cells or tissues of a host.

An “initiator cell” is a cell that gives rise to a cell or cells that are responsible, directly or indirectly, for creating a pathology, disease or disorder. A “causative cell” is a progeny of an initiator, either first or later generation progeny, that is directly or indirectly responsible for creating the pathology, disease or disorder.

“Chimerism” is the presence of at least one allogenic cell within an organism. The allogenic cells may circulate throughout the body or be largely restricted to a specific body location, or if capable, move from one location to another.

A “cell” is used herein to refer to a cell both in the singular and the plural forms unless the context clearly indicates otherwise.

“Host versus graft reaction” refers to the immune reaction of a host against allogenic or xenogenic cells acquired as a graft or otherwise. The reaction results in the damage or destruction of the grafted cells.

“Ectopic” refers to the abnormal location or position of a cell, tissue or organ.

“Oestrogen” refers to any one of a number of steroid hormones produced chiefly by the ovaries that are responsible for promoting estrus and the development and maintenance of female secondary sex characteristics.

“Developmental-self” describes the state of tolerance of a female host's immune response or the inability of the host immune response to distinguish between true “self” cells and closely related allogenic cells, for example those of a fetus. For example, the state of the maternal immune system during pregnancy is the developmental-self immune state, where the presence of non-self antigens does not trigger a host versus graft reaction against the genetically related developing embryo or fetus. This is due in part because specialized immune cells are at sentry where blood leaves the mother and enters the fetus, or leaves the fetus and enters the mother, and because the mother's CMI is down regulated, producing a hiatus in endometrioma resolution, as well as endometriosis symptoms, and is permissive of ESB expression of ESF cells, in somewhat the same fashion as anti-oxidants. In the “developmental-self” immune state, the host's cell-mediated immune response is attenuated but the host's humoral response is potentiated. The term “potentiated” is used herein to describe any measurable increase in a response. This down regulation of CMI may even be permissive of the ESB cell transfer between mother and fetus.

“Commensal-like” as used herein refers to a cell including an allogenic cell, or cells present at the time the fetus is editing T cells, which cell is thereafter accepted as a normal body cell for life by that infant.

“Sequential cell delete” describes a discovery process in which a potentiated immune system and non-invasive scanning of the body's interior assisted by appropriate recording and targeting software is used to identify the three-dimensional location of an allogenic ESB cell which creates, directly or indirectly, a pathology by altering body architecture. Sequential cell delete enables the extraction of ESB cells causing pathology and allows the genetic (including micro satellite and mitochondrial) or histocompatability profiles of these cells to be determined with the least damage to the patient's normal cells and tissues. Furthermore, such extraction may provide palliative relief to the patient from the symptoms of the pathology associated with the ESB cells.

“Cell edit” describes a curative process in which the host's immune system is manipulated to evoke a response against allogenic ESB cells that give rise to allogenic ESF cells that are responsible, directly or indirectly, for creating pathology. The process includes, for example, culturing allogenic ESB or ESF cells with the host's peripheral blood mononuclear cells to activate cytotoxic T-cells according to methods known in the art (Mutis et al, Biology of Blood and Marrow Transplantation (2002) 8: 412). “Peripheral blood mononuclear cells” include T-cells and antigen-presenting cells, for example B-cells mononuclear cells and dendritic cells. Peripheral blood mononuclear cells can be obtained from whole blood as would be known to a person skilled in the art. The activated T-cells, once reintroduced back into the host, will find and destroy allogenic ESB and ESF cells with the same genetic or histocompatability profile as those cells used to activate the T-cell ex vivo (Small et al (2001) J. of Clinical Oncology 18(23): 3894-3903). “Cell edit” also includes other mechanisms of inducing an immune response against allogenic cells, for example, by deliberately creating a local irritation in the area surrounding allogenic initiator or causative cells.

“Conceptus” describes a developing fetus or embryo, and a failed conceptus is a spontaneous failure of pregnancy in less than the first three months.

“Treating” a disease state refers to obtaining beneficial or desired results, including clinical results. Beneficial or desired results can include, but are not limited to, alleviation or amelioration of one or more symptoms or conditions, diminishment of the extent of disease, stabilization of the state of disease, prevention of the development of disease, delay or slowing of disease progression, delay or slowing of disease onset, amelioration or palliation of the disease state, and remission (whether partial or total), whether detectable or undetectable. “Treating” can also mean inhibiting the progression of disease, slowing the progression of disease temporarily, although more preferably, it involves halting the progression of the disease permanently. Treating may also mean preventing the agent responsible for the symptoms of the disease or condition from being transmitted to subsequent offspring.

“Stratum functional” cells or “endometrial stratum functional” cells refer to cells that constitute the outermost layers or mucosa of the endometrium closest to the uterine cavity. Some stratum functional cells are shed during each menstruation.

“Stratum basale” cells or “endometrial stratum basale” cells refer to cells that constitute the inner most layer of the endometrium, forming a membrane like barrier between the uterine muscle tissues and the ESF mucosa. These cells are retained during menstruation and provide a regenerative source for stratum functional cells and for themselves. These cells have amoebae like self-motile ability used normally during repair of this membrane like layer, for instance, after a pregnancy. A covert endometrial stratum basale cell is an endometrium stratum basale cell that is not associated with an endometrioma. Such cells may not produce ESF daughter cells until the CMI is down-regulated.

Without being limited to any particular theory, it is believed that endometriosis is caused by the presence of allogenic endometrial stratum basale cells in the afflicted individual. These stratum basale cells are adept at creating and directing growth of veins and arteries and supporting a large mass of daughter endometrial stratum functional cells. Stratum basale cells have strong stem cell capability, in that they are capable of producing daughter cells that are themselves incapable of reproduction and are physiologically different from their parent stratum basale cells, capable of structural change, capable of initiating or directing the construction of other tissues, capable of changing blood or capillary flow. Furthermore, the stratum basale and its stratum functional daughter cells are immunologically designed to accept, and not react to or with, an environment of somewhat foreign cells, for example, fetal cells during true pregnancy.

Without being limited to any particular theory, it is believed that allogenic cell apoptosis may increase the concentration of extracellular iron within an endometrioma, and that this excess iron may inhibit the ability of CMI (cell mediated immune system) cells to effectively deal with non-self cells and non-self cell remnants by reducing effective CMI phagocytosis. This prevents macrophages from making and presenting antigens to other CMI components. The source of the excess iron may also be from red blood cells whose capillary flow has been reorganized by an ectopic stratum basale cell to support these ESF daughter cells, some of which must be rebuilt after each such episode.

The immune system has evolved to distinguish between self and non-self antigens and to largely eliminate self-reactive lymphocytes. Because the repertoire of immune specificities is vast and largely random, it is not surprising that many nascent lymphocytes possess receptors for self-antigens. The mechanism of intrauterine tolerance is not well understood, but much has been learned about this mechanism for excluding or inactivating self-reactive lymphocytes, particularly by using the model of experimentally induced immune tolerance to foreign antigens (Baron, ed. Medical Microbiology, http://www.ncbi.nlm.nih.gov/books/bv.fcgi?call=bv.View..ShowSection&rid=mmed.section. 235).

When an antigen is introduced into an immunologically immature or newborn mammal, they may upon reaching maturity become unresponsive to immunization with that antigen (neonatal tolerance). This immunological tolerance is characterized by the absence of both antibody and cell-mediated responses, and it is specific for the original antigen (Baron, ed. Medical Microbiology, available online at http://www.ncbi.nlm.nih.gov/books/bv.fcgi?call=bv.View..ShowSection&rid=mmed.section. 235) providing a commensal-like status for cells containing, expressing or presenting such antigens.

The induction of antigen-specific tolerance is not always restricted to immature organisms. Unresponsiveness can also be induced in adults by using relatively higher doses of soluble antigen (high dose tolerance). The induced state of unresponsiveness to the antigen is sometimes accompanied by the appearance of suppressor T cells that actively and specifically inhibit the responses of B and T cells (Baron, ed. Medical Microbiology, available online at http://www.ncbi.nlm.nih.gov/books/bv.fcgi?call=bv.View..ShowSection&rid=mmed.section. 235).

Collectively, the experiments on tolerance induction demonstrate that the unresponsiveness to self is likely to be achieved at several levels. During normal development, the self-reactive lymphocyte clones may be inactivated or deleted by exposure to self macromolecules during the early stages of maturation in the thymus. This auto-selection is dependent upon MHC class I molecules for CD8⁺ T cells and class II molecules for CD4⁺ T cells. Those cells that are not eliminated and reach their full immunological potential may be inactivated when self molecules are presented to these cells at high concentrations or in a form that is tolerogenic rather than immunogenic. Also, it is possible that some self-reactive lymphocytes are suppressed by other regulatory cells, such as CD8⁺ suppressor T cells (Baron, ed. Medical Microbiology, available online at http://www.ncbi.nlm.nih.gov/books/bv.fcgi?call=bv.View..ShowSection&rid=mmed.section. 235). These processes create a kind of commensal-like status for those ESB cells present at this time, but not those ESB daughter ESF cells, which are not present until oestrogen cycling commences.

Mechanisms for creating microchimerism are known, and microchimerism in humans may not be a rare event (Adams et al., (2004), JAMA 291(9) 1127-1131). For example, maternal reabsorbance of embryonic or fetal cells may result in microchimerism in the mother (Khosrotehrani et al. (2003), Arthritis and Rheumatism 48: 3237). Reabsorbance may be triggered by the failed development of a single conceptus or after a sudden change in nutrient abundance, wherein one twin is reabsorbed in order to maximize nutrients available to the other twin (Lummaa et al., (1998) Nature 394, 533). In certain individuals, allogenic fetal cells may persist in the maternal body for decades after pregnancy (Bianchi (1996), PNAS 93:705). Mother to fetus cell trafficking, while occurring less frequently than fetal to maternal cell trafficking, has also recently been described (Nelson, Autoimmunity (2003), 36(1): 5). Alternatively, macrochimerism may result from the fertilization of two ova by two spermatozoa, followed by fusion of the zygotes and the development of an organism with intermingled cell lines (Neng et al, New Engl. J. Med. (2002) 346, 145). Of the mechanisms discussed, it is expected that the most frequent source of microchimerism is the partial maternal re-absorbance of a failed implanted conceptus less than 14 weeks old.

Only a percentage of such allogenic cell transfers are expected to involve endometrial stem or stratum basale cells. In order to account for the incidence of endometriosis, another mechanism is required. During pregnancy, the production of oestrogens by the maternal gonads is decreased and the fetal/placental unit is responsible for 90% of oestrogen production. The change in the site of oestrogen production during true pregnancy dispossesses the oestrogen-dependent self-motile endometrial stratum basale cells from the normally fluxing oestrogen-rich sites near the ovaries, and these stratum basale cells re-enter the bloodstream and lymph flow. Attracted by this local concentration of oestrogen at the fetal/placental unit, endometrial stratum basale cells cross the maternal-fetal blood barrier. Having crossed the maternal-fetal barrier into the placenta, the endometrial stratum basale cells have difficulty in crossing back into the maternal circulation, and may be incorporated into a developing fetus. This mechanism, described as “intergenerational contagion”, may provide a mechanism to increase the incidence of the intergenerational transfer of allogenic endometrial cells, and explains the whole body distribution of such cells in the infant and immature male or female. Such intergenerational spread of contagious disease is widely known (in AIDS, malaria, etc.) with respect to the transfer of both disease and parasite cells from mother to infant.

The first generation of allogenic endometrial stratum basale cells (and at menarche, ESF daughter cells of ESB cells) transferred by intergenerational contagion possess substantial genetic identity with their chimeric host (their mother, a successful conceptus carried by that mother, or that original cell's sister/brother during a succeeding pregnancy) and are therefore largely non-immunogenic. If this transfer takes place (for instance to a twin in the womb) before the process of fetal T cell editing, it may provide such ESB cells (but not ESF daughter cells) with a commensal-like status as a form of self cell. As these cells are serially transmitted to subsequent descendents, the genetic differences between these endometrial stratum basale cells and their host remains within the scope of commensal tolerance, but those ESF daughter cells not enjoying commensal-like status are increasingly more foreign. Since the advent of a reabsorbed conceptus is not that rare an event, there is likely to be more than one generation of such cells in any one host. In second or third generation hosts suffering stage I endometriosis, large ectopic cell volumes may still appear after menarche, but the non-commensal like ESF cells most foreign to their host are successfully resolved by their host, so that cell volumes, and immune cell numbers are seen to decrease in that host, until only those most genetically alike are left, and it is these “nearly self” residual colonies that now promote that noted autoimmune reaction, explaining why those individuals with the least exogenous endometrial endometriomas have the worst symptoms. These symptoms include a very high rate of spontaneous autoimmune-related abortion (Pathogenesis of Endometriosis: Natural immunity dysfunction or autoimmune disease? Guiseppe Matareses, et al, in “Trends in Molecular Medicine”, VOL 19, No 5, May 2003).

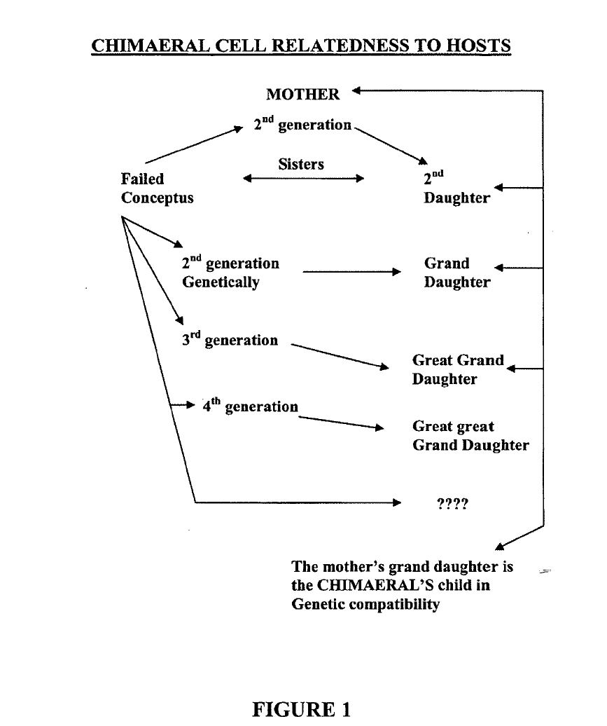

The genetic relationship of the parties in intergenerational contagion is depicted in FIG. 1. For example, if a woman becomes chimeric by reabsorbing a newly implanted conceptus, the surviving fetal ESB cells may be accepted, as previously noted, as a kind of commensal cell through known tolerance induction processes. Since their daughter ESF cells are also closely related to their first host, very little CMI reaction will result even though the daughter ESF cells were not accepted as commensal-like. This reabsorbed conceptus would be genetically first generation, the mother who bore them and acquired these cells being the more genetically different second generation, and any further daughters the same woman had with the same partner would be genetically first generation, closer genetically to the absorbed conceptus than to their mother. That is, any subsequent female children of a microchimeric mother would, in effect, be a “sister” to the reabsorbed conceptus, permitting CMI tolerance of those ESF cells. If this daughter had a daughter, that child would be second generation, the same genetic distance as her grandmother from these allogenic cells. In this fashion, a third generation allogenic ESB cell could still be tolerated and benefit from the immune state of neonatal tolerance and cell transfer described as a commensal-like state. As this status for this cell is renewed with each generational transfer, the ESB daughter ESF cells would not be as easily tolerated and may be resolved as a result of their host's CMI activities, producing a covert ESB cell group. In a normal open breeding pool, the allogenic cell may be transferred through 3 or 4 generations of female lineage by intergenerational contagion before provoking a strong host versus graft reaction, and even then, such a reaction would be to the ESF daughter cells, not the ESB mother cells. In more closed breeding pools, the allogenic cell may be transmitted for six or seven generations before its daughter ESF cells invoke a similar host versus graft response. Seven transfers appear to be the maximal limit before this altered immune state, which does not cover those ESF daughter cells, fails completely. In this way, this commensal like state of T cell editing during pregnancy reinforces prenatal and neonatal tolerance, also called fetal tolerance herein, for those ESB cells but not their ESF daughter cells. This situation creates a pool of covert ESB cells. This condition transmission route is ultimately limited by the observed decrease in fertility accompanying repeated generational increases in autoimmunity preventing or spontaneously aborting pregnancy, but this multi-generational effect transmitting ESB cells is enhanced by such early stage spontaneous autoimmune abortion, potentially creating new survivor ESB cells.

Without being limited to any particular theory, it is believed that fetal tolerance for maternal or other allogenic cells can promote lifetime tolerance to these somewhat foreign cells obtained during maternal/fetal or fetal/fetal cell trafficking granting a kind of commensal-like status to these ESB cells. This tolerance mechanism is specific, and is unlikely to be as tolerant of stratum functional cells as these cells did not exist in the pre and early post-natal period. These stratum basale cells may be acquired during the subject host's prenatal period from a twin sister (fetal/fetal cell trafficking), or from her mother (maternal/fetal cell trafficking of a cell acquired during the mother's infancy or a previous pregnancy). These stratum basale cells may be further transfused to a fetus of that woman, re-initiating fetal tolerance, or otherwise obtain a commensal like status in that subject during T cell editing for many generations.

This maternal/fetal trafficking may provide a preferred pathway for selected transmission of oestrogen-dependent endometrial stratum basale cells, which pathway may be enhanced by the known shift in oestrogen production from the ovaries (the center of the most common site of endometrial implantation) to the maternal/fetal compartment during pregnancy. This pathway may further explain the findings of tolerated endometrial cells in the infant, the male, pre-puberty female, and the adult female after hysterectomy and oophorectomy when subsequently undergoing treatments requiring oestrogen supplementation.

Male children will tolerate these very few allogenic endometrial stratum basale cells, unless the latter begin to produce stratum functional cells, for example, should the male host undergo oestrogen chemotherapy for reproductive cancers or for a sex change. The unexpected discovery of endometriosis in males and infants, as a statistical fraction of all males or infants, would be a tiny sample of all males and infants, hiding a much higher real incidence. This hidden source of allogenic endometrial cells may however, prime the male host for graft reactions under certain conditions.

Alternatively, males foreign to a closed gene pool may transmit genes broadening the range of developmental-self tolerance in relatively closed breeding pools, for example, such as in Iceland.

In the developmental-self or the commensal-like state, the allogenic stratum basale cells are largely non-immunogenic. In response to an oestrogen cycling signal, the ectopic basale stratum cells, like their eutopic counterparts, proliferate to produce a large number of daughter stratum functional cells. These stratum functional cells can form a cavity under a membrane, bulging or deforming the entrapping membrane and creating lesions, locally decreasing membrane lubricity, resulting in inappropriate ligatures joining adjacent tissues or ulcerations under tissue serous or mucosal membranes at ectopic sites when they undergo a near monthly apoptosis. The cycles of stratum functional proliferation and apoptosis may create ectopic blood pooling and generate cellular debris that cannot be cleaned up by the host's unpotentiated phagocytic cell-mediated immune response due to the immune system blocking capabilities of excess degraded heme and/or the commensal-like immune state preventing CMI resolution, as ESF cells apoptosis creates a kind of blood blister under a membrane resulting in an endometrioma which gradually enlarges each month.

The inhibitory effect of excess iron on macrophage function has been previously described (Harhaji et al, Clinical & Experimental Immunology (2004) 137(1):109-116) and attributed to the blocking of nitric oxide and hydrogen peroxide production. Macrophages are involved in iron scavenging, returning degraded heme for reconstruction and reincorporation into red blood cells. Iron scavenging, however, inhibits phagocytosis.

Normally, at an injury site or at a site of fast growing allogenic cells, the surrounding cells secrete cytokines to increase blood flow to this area, initiating an inflammatory response recruiting cells of the cell-mediated immune system, including neutrophils, which may be attracted to the increased blood flow or signals generated by the site self cells called cytokines. Neutrophils may invade the site of the injury or the site of fast-growing allogenic cells and may commence phagocytosis of whole or injured self-cells and any foreign cells at this site. Neutrophils that have phagocytosed whole or injured cells including foreign cells may undergo apoptosis after two or three days. Prior to undergoing apoptosis, these cells display a phosphatidyl serine signal on their surfaces. Such neutrophils may be phagocytosed by macrophages. The macrophages may recognize phosphatidyl serine on the surface of the pre-apototic neutrophil before apoptosis takes effect, which triggers the macrophage to consume the neutrophil, prior to the macrophage secreting signals that promote resolution of the inflammation and healing, such as for example, transforming growth factor beta. Macrophages may take antigens sorted from intact neutrophil contents and present these antigens to other components of the CMI and humoral immune system (HIS), where they may be used to train an antigen-specific humoral components, for example B cells, as well as complement, and go through a process of providing “memory” to resident macrophages for future use. The administration of antibiotics to a host may impair this normal cell-mediated/humoral immune systems activity, and surgery subsequent to this event may remove the local population of resident macrophages maintaining memory of this event. The presence of degraded heme may interfere with this process until the source of Fe moieties is cleaned up. Improper or delayed clearance of apoptotic cells by phagocytes may result in autoimmune disorders (“Immunological Consequences of Macrophage-Mediated Clearance of Apoptotic Cells”, Aunjung, Kim, et al, in Cell Cycle. 2005 Feb. 7; 4(2)).

Alternatively, when the normal neutrophil signaling fails, for example, because of oxidative alteration or the presence of excess iron, neutrophils may undergo necrosis. Intracellular iron and degraded heme products may be particularly concentrated in an endometrioma. Neutrophil necrosis may cause surrounding macrophages to secrete a signal molecule that enhances inflammation, for example transforming growth factor alpha. Self cell-inflammatory factors that were consumed by the neutrophils, may be released into the extracellular space, further enhancing local inflammation. This further increases the local extracellular iron concentration, as blood flow may pool in a growing circumscribed cavity under a membrane as tissue ulceration continues to form and grow in these anoxic conditions.

When a macrophage consumes remnants of neutrophil necrosis, no antigens are preserved that can be presented to the humoral immune system. The ability of macrophages to present antigens to cells of the humoral immune system may be compromised when the host is dealing with commensal-like organisms as it is with opportunistic infections, or may be compromised in the presence of excess heme. This reduced antigen presenting activity is involved in producing a sustained inflammatory condition, and may continue after parturition while the developmental-self state of the immune system is returning to normal (and is thus permissive of new commensal/commensal-like or non-commensal microbial infections). Such reduced antigen presenting activity may also be involved in producing and maintaining the first endometrioma ESB cell infection in an adult human host, before sufficient iron is accumulated to protect those ESF cells.

Excess iron released from degraded heme by necrosis of blood cells is normally scavenged by macrophages as a part of an iron conservation mechanism in mammals. This iron regulation may be observed when macrophages invade the uterus in menses. Iron loading of macrophages has been shown to impair the ability of macrophages to respond to various inflammatory stimuli (Ward et al, (2002) Biochemical Society Transactions 30(4):762-765). Without being limited to any particular theory, it is believed that excess iron may reduce the effectiveness of the normal myloperoxidase oxidation routes of the cell-mediated immune system, impairing the ability of the cell-mediated immune system to manufacture their normal biocidals and engulfed matter digestive compounds, and thus preventing the previously explained pathway to resolution and healing of inflammatory conditions.

The iron scavenging and phagocytic activities of macrophages are mutually incompatible, with each pathway inhibiting the other. To some extent, the presence of excess degraded heme may result in an immune state similar to developmental self, or to the state of a commensal infection, as both prevent antigen presentation to the cell-mediated immune system and to the humoral immune system.

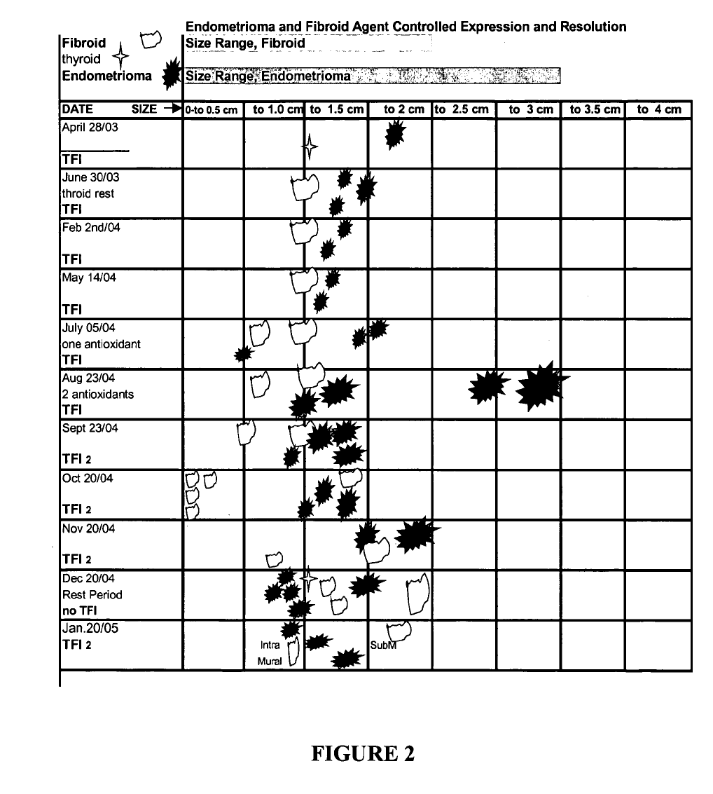

As outlined above, ectopic growths of allogenic stratum functional cells are generally non-immunogenic in the presence of excess degraded heme because macrophages can not present antigens resulting from allogenic stratum functional cell apoptosis to the humoral immune system or to helper T-cells and B cells of the cell-mediated immune system. However, the cell-mediated immune response may be potentiated, for example by the administration of an effective amount of iodine. Ghent et al (U.S. Pat. No. 4,816,255) disclose that elemental iodine can be used in the treatment of fibrocystic dysplasia. Clinical tests (FIG. 2) have demonstrated that the administration of iodine is effective in the clean-up of endometrial cell debris, aberrant organ attachments, endometriomas and stratum functional cell masses in patients. Similarly, excess heme may be additionally dealt with by using suitable chelating agents, for example desferrioxamine, which allow the cell-mediated immunity cells to operate more normally. Alternatively, such excess heme may be dealt with using apoptosis mediators extending the time between one menstrual period and another, to extend the time for macrophages to scavenge iron.

Without being limited to any particular theory, it is believed that iodine enhancement of cell-mediated immunity cells, allows these cells to scavenge iron by reducing it to FeI₂ independently as this reaction does not depend upon oxidation, but is a reducing reaction. Without being restricted to any particular theory, the use of iron chelating agents and iodine enhancement of macrophage iron scavenging would aid in preventing excess degraded heme from being oxidized by, or iron otherwise crippling biocidal oxidizers produced by myloperoxidase oxidation routes of the cell mediated immune system, allowing cleanup of this cellular debris to restart. This may well further enhance cell mediated immunity activity by allowing presentation of ESF cell antigens to the humoral immune system in vivo. Ex vivo presentation of ESB cell antigens is also possible, thereby avoiding the effect of suppressor T-cells that may be generated during the establishment of fetal tolerance or other commensal-like states.

In the potentiated state, after the excess iron at an endometrioma is reduced, the host's immune system becomes capable of distinguishing ectopic allogenic cells from true “self” cells. This is apparent from the normal immune systems' resolution of most endometriomas even when not potentiated without any effect on those normal self endometrial cells of the uterus. That is, the administration of an effective amount of iodine clearing the excess iron permits the host's immune system to selectively mount an immune response against residual ectopic allogenic ESF cells remaining that are too persistent for normal immune system activities. This enhanced state increases cell-mediated immunity such that phagocytic cells, particularly macrophages, can clean up both cellular remnants and active tissues anywhere in the body causing a pathology by distorting body architecture and creating a persistent inflammatory state in the presence of excess iron from degraded heme. This distortion of body architecture results from local swelling which is the necessary first step of an immune response. The swelling may be caused by invasive tissue growth and self cell reactions to that growth. This ability extends particularly to those tissues of ectopic endometrial stratum functional cells and any cellular debris resulting from stratum functional cell apoptosis, including inappropriate tissue attachments resulting from these endometrium cells crowding normal body architecture, and particularly endometriomas. This cleanup includes all such cells, including some cancer cells, in ectopic locations causing a pathology attractive to the immune systems in a similar fashion.

When the immune response is potentiated to scavenge iron as degraded heme, the immune system does not abnormally affect eutopic endometrial cells, ectopic cell debris not creating a pathology or ectopic allogenic stratum basale cells, since the latter do not cause a change in body architecture, have a commensal like status and, as a result, do not present a pathology attractive to the host's immune system. When not potentiated, this cellular debris remains trapped under a serous or mucosal membrane forming an endometrioma or cyst-like structure resembling a blood blister, sometimes called a chocolate cyst.

The amount of extremely high purity iodine as thermodynamically free iodine administered should be an amount in excess of normal body and dietary needs and which is effective in potentiating the host's immune response and can be readily determined by a person skilled in the art. The thermodynamically free iodine may be administered in water or other suitable carrier and may be accompanied by adjuvants such as anti-inflammatory agents, anticoagulants, anti-platelet agents and apoptosis mediators. As used herein, “anti-platelet agents” includes, for example, acetylsalicylic acid. For example, where administered as an aqueous solution of elemental iodine, the elemental iodine may be administered in the range of about 0.1 to about 28 mg per day per 50 kilograms of body weight above the host's dietary iodine requirements. The amount of iodine administered may be increased to about 0.2 to about 40 mg of iodine per day in excess of normal body and dietary requirements per 50 kilograms of body weight if necessary. The use of iodine to potentiate the immune response may be most effective if the regimen of administration is periodically interrupted or stopped. This may result in decreasing the amount of iodine necessary to potentiate the immune response, and to allow the thyroid to discharge excess iodide.

Differential immunogenicity of those cells in an endometrioma is demonstrated by the selective manner in which the CMI deals with them. Irrespective of size or number, a decrease in endometriomas is seen as a natural consequence of CMI activity in endometriosis patients. It is likely that the most foreign cells are cleaned up first, while those most genetically closely associated with their host and hence most likely to cause auto-immunity are cleaned up last. This clean-up of ESF cells of differential immunity results in some ESB cells not being able to express daughter ESF cells without attracting an immediate immune response, while those as yet unresolved continue to express daughter ESF cells. These ESB cells unable to express ESF cells form a population of covert ESB cells shown to exist in our first human clinical trial, as shown in FIG. 2.

The differential immunogenicity of the allogenic ectopic stratum basale cells affords a new method of identifying the cells that are ultimately responsible for the symptoms of endometriosis. By exploiting the different immunogenicity of allogenic endometrial stratum basale cells, the positions of the cells responsible for the symptoms of endometriosis may be identified. Whereas identifying and removing the ectopic stratum functional cells may provide some temporary palliative relief, this approach will not cure endometriosis as new stratum functional cells may be derived from ectopic stratum basale cells. In order to provide a more permanent cure for endometriosis, the allogenic, self-motile endometrial stratum basale cells must be removed or destroyed. In the context of endometriosis, eliminating the ESB cells may not only cure the previously afflicted host, but may also prevent the intergenerational contagion to the host's subsequent offspring, including males, or through blood transfusion, passing this contagion to unwitting strangers, including males, limiting the use of oestrogens for the treatment of disease in later life.

In one embodiment, the invention therefore provides a method of identifying an ectopic allogenic ESB cell. The method includes administering iodine to a host afflicted with endometriosis. The form of iodine may be any form that will act to potentiate the host's cell-mediated immune system such that the host's cell-mediated immune system can enhance macrophage ability to scavenge degraded heme, and thus allow normal phagocytosis of foreign and injured cells, permitting such cells to be engulfed by neutrophils, and neutrophils by macrophages in a timely fashion to provide material for antigen creation and presentation. Preferably, the form of the iodine is diatomic iodine, which has the least toxicity of all forms of iodine (Ghent et al (1993), Canadian J. of Surgery 36:453; and U.S. Pat. No. 4,555,347 (issued to O'Dowd)). The diatomic iodine may be in aqueous solution as disclosed in U.S. Pat. No. 4,555,347 to O'Dowd. The iodine may be administered orally, by breathing as a vapour, implanted internally, applied externally in a controlled release format, inhaled through an iodine vapour permeable membrane or through a temperature sensitive iodine permeable controlled release barrier as a dermal patch, or formulated as a pill with Halon starch. In the case of old and persistent endometriomas containing large volumes of degraded heme, specific human tolerant chelating agents may be administered to the endometrioma as well to allow the normal operation of cell-mediated immunity by preventing further oxidation but not reduction of that volume of iron present. As well, apoptosis mediators or other means such as oestrogen cycle suppressors may be used to extend the time between menstrual periods allowing the macrophage to clean up as much iron as possible before a new influx of degraded heme from ESF apoptosis appears in an endometrioma.

The exact position of the potentiated immune response against allogenic stratum functional cells and their residues may be determined by comparing a non-invasive soft tissue image taken before and approximately 28 days after iodine administration, noting the size reduction of the endometrioma. Upon ceasing such administration, the regrowth pattern of such stratum functional cells from the stratum basale mother cells may be observed by comparing the images. Regrowth confirms that ESB cells are present and active. The non-invasive imaging may be performed on the whole of the individual's body, or preferably on the abdominal or peritoneal cavity or other location where endometriosis has been proven. The location or locations of the potentiated cell-mediated immune response increasing degraded heme scavenging to allow antigen presentation and a subsequent immune response against the allogenic stratum functional cells can then be determined and plotted by comparing the images acquired before and after the immune system was potentiated. On ceasing immune system potentiation, the endometrioma will regrow very rapidly, if it is associated with active ESB cell(s).

Where these ESB cells are covert, CMI immuno-down regulators will be permissive of their re-appearance and cessation of those CMI immuno-down regulators in combination with iodine chemotherapy will allow these new endometriomas to resolve again as noted above and demonstrated in FIG. 2.

Any non-invasive imaging method may be used, for example, ultrasound, CT or x-ray radiography or MRI (Zawin et al, Radiology 171:6931). Although the size of endometriomas are expected to vary, the imaging means should be capable of detecting differences in lesion surface areas of at least about 0.1 cm². Ectopic endometrial lesions may be distinguished from non-endometrial lesions on the basis that endometrial lesions sequentially grow over a period tied to menstruation, have a different density than other lesions when contained and not treated and often have a characteristic colour from the degraded heme they contain, being referred to as a chocolate cysts.

The location of the cell-mediated immune response may be determined by the reduction in the size of an ectopic endometrial growth in the soft tissue image after iodine administration. The location of this cell-mediated immune response identifies the position of allogenic stratum functional cells. Alternatively, this location of allogenic stratum functional cells, and the presence of active associated ESB cells may be identified by an increase in ectopic endometrial cell mass in a soft tissue image collected after iodine administration has been suspended, or following CMI down-regulation, for example iatrogenic down-regulation of the CMI. Once the position of the allogenic stratum functional cells is known, and that the condition causative ESB cells are present and active by permitting them to re-grow ESF cells, the ESB cells may, if appropriate or necessary, be removed by needle biopsy or by laparoscopy. Alternatively, these cells may be irritated in a manner sufficient to induce an immune response against the commensal-like ESB cells. For example, where appropriate, chemical irritants, for example those found in poison ivy or poison oak, may be implanted to invoke a further local inflammation in the area surrounding the identified stratum basale cells and to initiate a further immune response against these cells.

Even when potentiated, the “commensal-like status” or heme-blockaded cell-mediated immune response may not identify the non-immunogenic allogenic basale stratum cells from which the functional cells are derived. A stratum basale cell may remain associated with its daughter stratum functional cells, in which case the location of the stratum basale cell may be determined from the position of the stratum functional cells' pattern of monthly regrowth. Alternatively, a self-motile stratum basale cells may move away from its stratum functional daughter cells, (in which case the stratum functional cells will not grow and be replaced beyond the next menstrual period). The position of the stratum basale cell may also be calculated by using a targeting algorithm, including software known to persons skilled in the art, for example a purpose-modified ENEAC ballistic targeting software program after demonstrating that it is associated with the endometrioma. The ENEAC ballistic targeting software has been demonstrated to accurately model and describe the sequential reversal of a pathway. Alternatively, the tracking software may be adapted from known software capable of tracking a baby's heartbeat as it travels through the birth canal, using a series of viewings through resolution and re-growth as the data signal, rather than the position of a sound as a data signal.

Once the location of at least one or more ectopic stratum basale cells are identified, such a cell may be removed from the body by minimally invasive normal surgical biopsy. While this may also be accomplished by normal surgical means during removal of an endometrioma, followed by specific identification of the stratum basale cell, followed by ex vivo culturing of the surgically removed stratum basale cell or cells, the use of minimally invasive biopsy techniques may minimize consequential damage to the surrounding tissues. This alternative means allows the size of the biopsied tissue to be kept as small as is practical while ensuring that a stratum basale cells is removed. In a preferred embodiment, the allogenic basale cell is removed using an apparatus for automated biopsy and collection of soft tissue such as that disclosed in U.S. Pat. No. 5,980,469.

Identifying the location of the stratum basale and stratum functional cells allows these cells to be removed from the host and the genetic or histocompatability profiles of these cells can be determined. The allogenicity of the ectopic endometrial cell, relative to other “self” stratum basale cells obtained from the uterus of the host being treated provides a diagnosis of endometriosis.

In another embodiment, the invention provides a method for identifying endometrial stratum basale cells that are not associated with an endometrioma, referred to as covert stratum basale cells. The method comprises the step of administering an effective amount of an agent that down regulates cell-mediated immunity, such as, for example, antioxidants. As used herein, “antioxidants” include, but are not limited to, vitamin E, zinc and their combination, as well as other iatrogenic immune system down regulators. Without being limited to any particular theory, it is believed that under normal states of cell-mediated immunity, some of the ectopic stratum basale cells are covert stratum basale cells whose daughter stratum functional cells have been successfully targeted and resolved by the CMI. Under conditions of decreased cell-mediated immunity, these ectopic covert stratum basale cells may be induced to express stratum functional cells allowing an endometrioma to form. The endometrioma thus formed (and the formerly covert ectopic stratum basale cell), may be detected or treated according to different embodiments of the invention.

Down regulators of cell-mediated immunity may be administered according to methods known in the art, including orally, or, by implantation, for example as a time/volume release reservoir or iatrogenically activated reservoir, within the peritoneal cavity. The effective amount to be administered to a patient can vary depending on many factors such as, among other things, the mode of administration, the age, health and weight of the subject, the nature and extent of the disorder or disease state, the frequency of the treatment and the type of concurrent treatment, if any. For example, for oral administration, vitamin E may be administered as alpha tocopherol at a dosage of approximately 800 IU day and 50 mg zinc, as soluble zinc, may be administered at a dosage of about 50 mg/day per 50 kg of body mass in a female. The effective amount of an immuno-down regulator of cell-mediated immunity to be administered may be determined by a person skilled in the art.

In another aspect, the invention provides a method of treating endometriosis. In one embodiment, allogenic stratum basale cells are removed from the host afflicted with endometriosis. The cells may be removed from the body by a number of different methods. For example, one or more cells removed by biopsy may be presented to a culture of the host's peripheral blood mononuclear cells. Ex vivo, the cultured T-cells and the commensal-like status of the ESB cells is thus revoked, as has been shown in opportunistic infections (“Possibilities For Active and Passive Vaccination Against Opportunistic Infections”, http://www.isgnas.org/doc/meeting_summary.html).

T cells may be trained to immunogenically recognize allogenic basale stratum cells. The basale stratum cells may then be used to activate a subset of the host's T cells. Induction of a cell-mediated immune response may be readily assessed by testing for the presence of cytotoxic T-cells in the ex vivo culture, for example using the standard CTL assay known in the art. The activated T cell population of the ex-vivo cell culture may be reintroduced back into the host organism, for example by being transfused back into the patient. Once reintroduced (re-transfused) back into the host, the ex-vivo activated T cells will selectively destroy stratum basale cells that express the same surface antigens as the extracted basale stratum cell while at the same time causing little or no damage to surrounding “self” tissues and cells, including the normal self cells of the uterus. This procedure may be repeated as necessary to destroy basale stratum cells with different histocompatabilities or genotypes. The ESB cells may also be removed by surgery.

In another embodiment, the in vivo immune response produced by ex vivo trained T cells may be enhanced by transfusion of a live transgenic vaccine, for example a pox-virus, that has been genetically engineered to express an allogenic stratum basale cell-specific antigen wherein the antigen has been recovered from a host's endometrioma, or an immunogenic fragment thereof. Without being limited to any particular theory, it is believed that the introduction of a transgenic virus expressing an allogenic stratum basale cell specific antigen will create an immediate response by the host's humoral immune system.

In yet another aspect, the invention provides a method of diagnosing endometriosis comprising comparing the host genotype (i.e. that of the host “self” cells) to the genoytype of an ectopic endometrial cell. The allogenicity of the ectopic cell confirms endometriosis. The ectopic endometrial cell may be an ESB cell identified according to the present invention. Genotyping, including polymorphisms and microsatellite DNA analysis, of the respective cells may be accomplished by methods known to a person skilled in the art, for example restriction fragment length polymorphism (RFLP), single length polymorphism (SNP), or Short Tandem Repeat (STR) assays.

As can be understood by one skilled in the art, many modifications to the exemplary embodiments described herein are possible. The invention, rather, is intended to encompass all such modification within its scope, as defined by the claims.

### EXAMPLES

The term “TFI” refers to thermodynamically free iodine and the term “TFI²” refers to thermodynamically free iodine used in combination with an adjuvant, such as an apoptosis mediator. In the results discussed herein, TFI² included treatment with the apoptosis mediator, aspirin. “Counter therapy” refers to treatment involving use of compounds that down-regulate the CMI, such as antioxidants. Antioxidants used here were vitamin E and zinc.

Although the ESB cell is commensal-like and not recognized by the CMI, endometriomas seemed to be resolved in a sequential manner, possibly for one of the following four reasons:

(i) the ESF cells have sufficient difference in genetics from their host and are unprotected by the commensal state so that the CMI attacks them when possible, but is usually prevented by iron homeostasis blocking macrophage antigen creation and presentation;

(ii) for some reason under some rare conditions at the start of the monthly grow-out cycle some random group of ESF cells become unprotected, for instance, because iron has coagulated into a solid scab which the rapid growth of these cells pushes up and out, allowing CMI cells to get underneath this iron laden scab and attack these growing ESF cells, thereby sending a valid antigen to the appropriate CMI cells, which then successfully attack these cells;

(iii) the parent ESB cells occasionally move and do not produce daughter cells for some unknown reason, until treatment with thermodynamically free iodine (TFI) is stopped, when they return to their previous locations; or

(iv) the CMI was otherwise down-regulated, permitting these commensal like ESB cells to produce non commensal like ESF cells.

We suggest that TFI enhancement of the macrophage allows the production of FeI₂, a soluble not easily re-oxidizable iron scavenged by the macrophage by reduction from FeO, or FeO₂ producing FeI₂ and that this permits a net reduction in oxidation states of all iron at a site, allowing iron from degraded heme to be taken up by macrophage scavenging (as it is now soluble and disassociated from other elements) in a kind of cascade effect. This can be achieved by TFI because it is an oxidizer that makes its own oxygen, reducing water, H₂O to HIO+H, and a reducing agent for other oxidized materials (by I₂, or HIO), when that requires less energy and so does not require effort on the part of the CMI cell needed to work at oxidation of water to H₂O₂ in anoxic conditions, which product, as would NO, just further oxidize Fe anyway.

When TFI chemotherapy is stopped to allow the thyroid a rest, we see a general improvement in iron scavenging at lower dose rates afterwards (which hints at a multi-step process that can be slowly poisoned, most probably by I⁻ to tri-iodide) and usually a recovery to visibility in those in-completely resolved endometriomas during this treatment hiatus. We timed this 3rd party evidence trial to happen as we approached a pre-planned thyroid rest period, taking monthly ultrasounds before to position each active endometrioma as closely as possible, and include three such rest periods, plus two thyroid examinations.

In this process, which is an enhancement of the natural process, the most dramatic drop in the number of endometriomas happens in the first year, Stage I Endo, and then the process slows down. This resolution gradient is thought to be caused by helping the CMI to get to the most foreign ESF cells first, by quickly resolving the Fe problem at many small endometrial implants, but at stage IV things slow down. The endometrioma in the left ovary has proven the most difficult to get to, and is probably the most genetically like its host.

The simplest test was to gently down-regulate the CMI in a patient while that patient was still on TFI chemotherapy, and to observe what happens next. The patient then took 800 international units of vitamin E a day (as a tocopherol). Minor change was observed, with new endometriomas developing and old ones rapidly increasing in size, but the result was not as dramatic as expected. A second antioxidant, dietary zinc (10 mg/day), was added to the regimen. Use of both antioxidants allowed us to say that this was not a special case for either antioxidant, though the cumulative effects were very obvious. Vitamin E was stopped two weeks before zinc, to provide a period of zinc only. TFI-induced resolution was still blocked. We had many more endometriomas in new locations and of very large size by the end of the 2nd month, so we cut off all antioxidants and continued with TFI, adding an apoptosis regulator, aspirin, to further damp down the effects of the antioxidants. A steady rapid reduction in endometriomas in size and number brought the patient back within her normal range quickly.

The patient then went into her thyroid rest period, and as usual a few recently but incompletely resolved endometriomas developed, but so did two that had been thought resolved from two years ago for a total of five.

In using transvaginal ultra sound, it was possible to view and compare the thickness of the uterine lining, and no abnormal change was observed despite all the activity in the peritoneal cavity.

Thus, we have shown that the successful CMI-driven self-resolution of the endometrioma is the norm in Stage I to Stage IV endometriosis, and probably accounts for all such resolution up to the mid or end of Stage III, but at Stage IV this successful resolution stalls. The use of TFI speeds this multi year (which otherwise takes about 15 years) process up, reducing such resolution to about one or two years, and provides relief from pain. We have also shown that self-resolution, and TFI resolution as such, is most likely only resolution of the non commensal-like ESF cell, in that we can create a condition (by immuno-down regulating) under which these hidden ESB cells can again start production of ESF cells.

We suggest that the effect of vitamin E, zinc and likely other OTC antioxidants on endometriosis is to decrease CMI capability, allowing a return to the asymptomatic state. It appears that these products may reduce symptoms and that they may do so by reducing CMI capability and thereby unacceptably increasing patient risk. This may be similar to what happens during pregnancy; that is, the CMI is down-regulated so symptoms decrease in the absence of menstrual cycling.

Antioxidants and pregnancy do reduce patient symptoms as the symptoms are generated by blocked immune activity, a kind of immuno-frustration. The remaining endometriomas at stage IV are those most difficult to resolve likely because they are those most closely related to their host. In learning to attack not quite self cells, these CMI cells are coming too close to attacking self, and this is likely the cause of spontaneous autoimmune abortion seen accompanying endometriosis at this stage. Those last endometriosis ESB cells and their ESF daughter cells may be at least as genetically close to the patient as a fetus of the patient, and perhaps even as close as a twin of the patient. This patient's familial history would lead us to suspect it is a sister's cell, but we will not know until it is geno-typed.

This clinical demonstration has provided deductive evidence to support our thesis of allogenic cell contagion pathways providing multi generational contagion of resident commensal-like ESB cells in patients.

This method can be used to test after surgery to demonstrate effectiveness of surgery without the need to wait five years. This method can also be used in a method of harvesting cells for geno-typing/re-transfusion to catch all the endometriomas no matter where they are located in the body.

The patient in the above study had too many endometrial implant sites to count, and was in Stage I when diagnosed. It is probable that almost all of those ESB cells, in their hundreds, are still potentially active, but currently covert. Any immuno-down regulator will work to permit these commensal like cells to re-activate.

It should be noted that use of counter chemotherapy produced no change in those similar normal self-cells of the uterus as detectable by ultrasound. All of these ultrasounds were taken at 3 to four days after the patient's periods, except the last one as she had her period cycle extended by taking aspirin.

1. A method for identifying an ectopic allogenic initiator cell in a mammalian host, the method comprising:
a) obtaining a first internal soft tissue image;
b) administering an effective amount of iodine to potentiate the host's immune response to aberrant ectopic tissue;
c) obtaining one or more subsequent internal soft tissue images after said administration; and
d) comparing the first internal soft tissue image and the one or more subsequent internal images to identify the position of the ectopic allogenic cell.

2. The method according to claim 1 wherein the ectopic allogenic initiator cell is an endometrial stratum basale cell.

3. The method according to claim 1 wherein the method is a method for treating endometriosis:
wherein the ectopic allogenic initiator cell is an allogenic endometrial stratum basale cell, the method further comprising
removing the ectopic allogenic endometrial stratum basale cell.

4. The method according to claim 3 wherein removing the allogenic endometrial stratum basale cell comprises irritating the endometrial stratum basale cell in a manner sufficient to induce an effective immune response against the endometrial stratum basale cell.

5. The method according to claim 4 wherein said irritating comprises adding a bio-irritant or chemical irritant to or near the endometrial stratum basale cell.

6. The method according to claim 5 wherein the chemical irritant is derived from poison ivy or poison oak.

7. The method according to claim 3 wherein removing the allogenic endometrial stratum basale cell comprises:
a) removing one or more ectopic allogenic endometrial stratum basale cells from the host;
b) stimulating a cell-mediated immune response by reacting the allogenic cell with the host's immune cells ex vivo; and
c) reintroducing the host's immune cell into the host's body to effect a cell-mediated immune response in vivo against the allogenic stratum basale cells in the host.

8. The method according to claim 7 wherein the host's immune cells are peripheral blood mononucleocytes.

9. The method according to claim 1 wherein the ectopic allogenic initiator cell is an allogenic ectopic causative cell.

10. The method according to claim 9 wherein the allogenic ectopic causative cell is an endometrial stratum functional cell.

11. The method according to claim 1 wherein the method is a method for determining or improving the efficacy of treatment of a pathology, disease or disorder caused by the presence of allogenic commensal or commensal-like cells in a mammalian host, wherein the first internal soft tissue image is obtained subsequent of said treatment and wherein said ectopic allogenic initiator cell is a remaining ectopic allogenic initiator cell or an ectopic allogenic causative cell.

12. The method according to claim 11 wherein the treatment comprises surgery.

13. The method according to claim 12 wherein surgery comprises laproscopy.

14. The method according to claim 1, further comprising administering an effective amount of a down-regulator of cell-mediated immunity prior to obtaining the first internal soft tissue image.

15. The method according to claim 14, wherein the down-regulator of cell-mediated immunity is an anti-oxidant.

16. The method according to claim 15, wherein the anti-oxidant is vitamin E, zinc, or a combination thereof.

17. The method according to claim 1 wherein the effective amount of iodine is from about 0.1 to about 28 mg/day in excess of the host's normal body and dietary requirements.

18. The method according to claim 1 wherein the iodine is diatomic iodine.

19. The method according to claim 18 wherein the diatomic iodine is administered in aqueous solution.

20. The method according to claim 1 wherein the first internal soft tissue image and the one or more subsequent internal soft tissue images are obtained by a non-invasive imaging method.

21. The method according to claim 20 wherein the non-invasive imaging method is ultrasound, magnetic resonance imaging, computer tomography or x-ray radiography.

22. The method according to claim 1 wherein the position of the allogenic initiator cell or the allogenic causative cell is identified using a targeting software.

23. The method according to claim 22 wherein the targeting software is a purpose-modified ENEAC ballistic targeting program.

24. The method according to claim 22 wherein the targeting software is adapted from software developed to measure the movement of a baby through the birth canal.

25. The method according to claim 1 further comprising administering an effective amount of an adjuvant in combination with the iodine.

26. The method according to claim 25 wherein the adjuvant is an iron-chelating agent.

27. The method according to claim 26 wherein the iron-chelating agent is desferrioxamine or o-phenanthroline.

28. The method according to claim 25 wherein the adjuvant is an apoptosis mediator, an anti-platelet agent or an anti-inflammatory.

29. The method according to claim 28 wherein the apoptosis mediator is acetylsalicylic acid.

30. A method of diagnosing endometriosis in a mammalian host, the method comprising comparing the host's genotype to the genotype of an ectopic allogenic endometrial stratum basale cell or an ectopic allogenic endometrial stratum functional cell.

31.-57. (canceled)
